(19) United States
(12) Patent Application Publication
(10) Pub. No.: US 2009/0149461 A1
(43) Pub. Date: Jun. 11, 2009

(54) METHOD OF TREATING CANCER

(75) Inventor: Andrew P. Krivoshik, Abbott Park, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/328,992

(22) Filed: Dec. 5, 2008

Related U.S. Application Data
(60) Provisional application No. 61/058,113, filed on Jun. 2, 2008.
(60) Provisional application No. 60/992,857, filed on Dec. 6, 2007.

(51) Int. Cl. A61K 31/5377; A61P 35/00

(52) U.S. Cl. 514/235.5

(57) ABSTRACT

Methods of treating cancer using N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide are disclosed.

### CROSS REFERENCE TO RELATED APPLICATION

The present application claims priority to U.S. Provisional Application Ser. No. 61/058,113, filed Jun. 2, 2008; U.S. Provisional Application Ser. No. 60/992,857, filed Dec. 6, 2007; hereby incorporated their entirety by reference.

### FIELD OF THE INVENTION

This invention pertains to methods of treating cancer using N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide.

### BACKGROUND OF THE INVENTION

Anti-apoptotic Bcl-2 family protein members are associated with a number of diseases and are therefore under investigation as potential therapeutic drug targets. These important targets for interventional therapy include, for example, the Bcl-2 family of proteins Bcl-2, Bcl-X_{L} and Bcl-w. While this art teaches inhibitors having binding to the target protein, this is only one of many parameters that must be considered as a compound is investigated for further or continued drug development. As part of this development, it is highly desirable to have compounds that are orally efficacious in mammals and have tolerable side-effects profiles, the nature of which are preferably determined by administering to mammals and determining the side-effects and severity thereof.

Accordingly, there is an existing need in the therapeutic arts for efficacious cancer chemotherapeutics with tolerable side effects profiles.

### BRIEF DESCRIPTION OF THE FIGURES

FIG. 1 is a graph showing mean ABT-263 plasma concentrations during dosing at 315 mg.

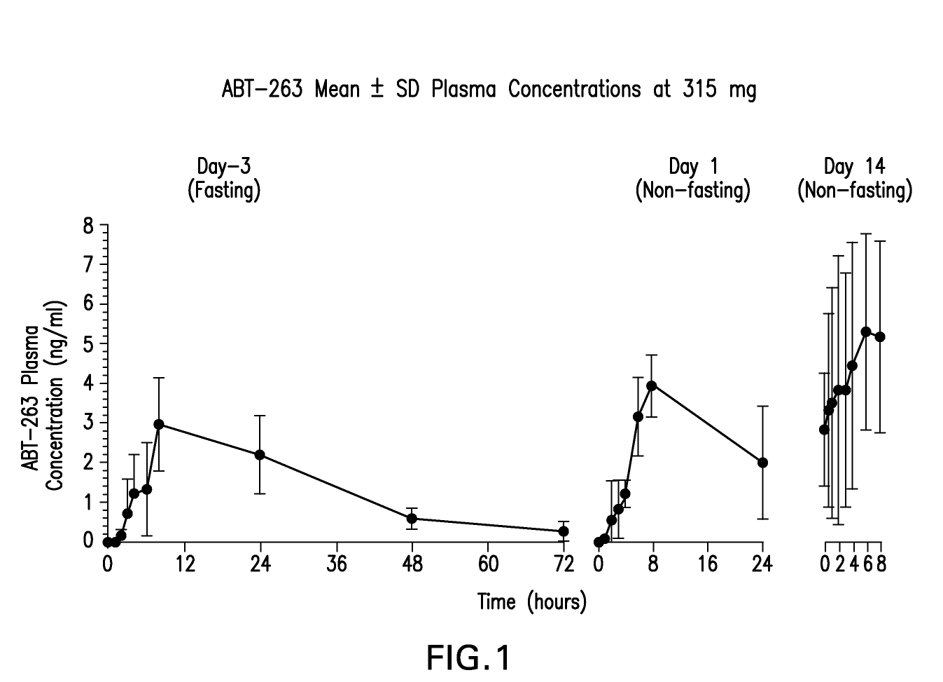

FIG. 2 is a graph showing dose proportionality under fasting and non-fasting conditions.

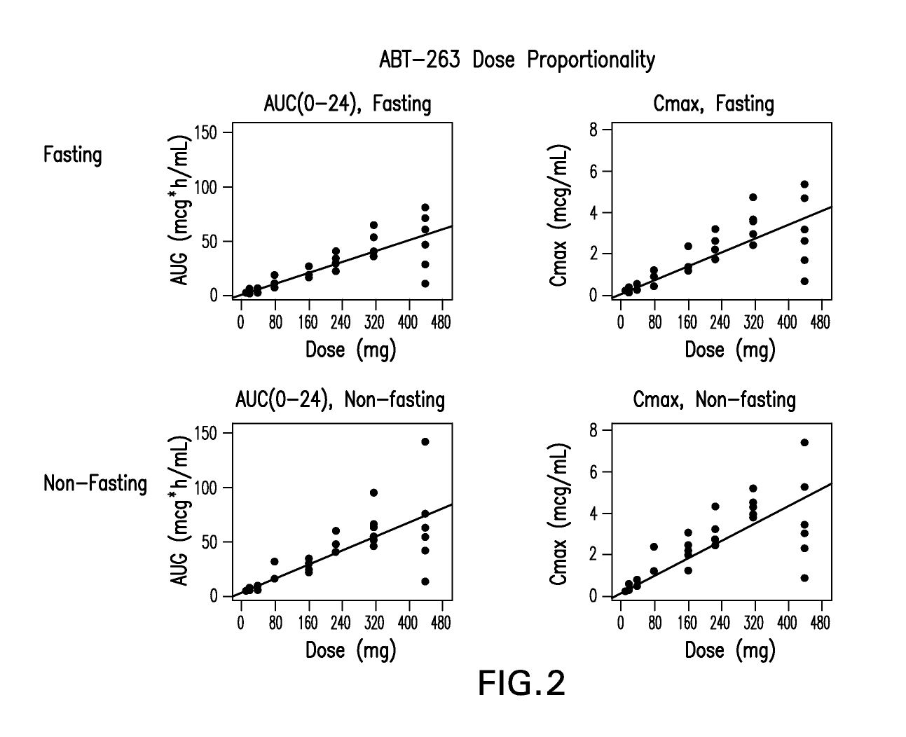

FIG. 3 illustrates effect of ABT-263 on platelets at different doses over several dosing cycles.

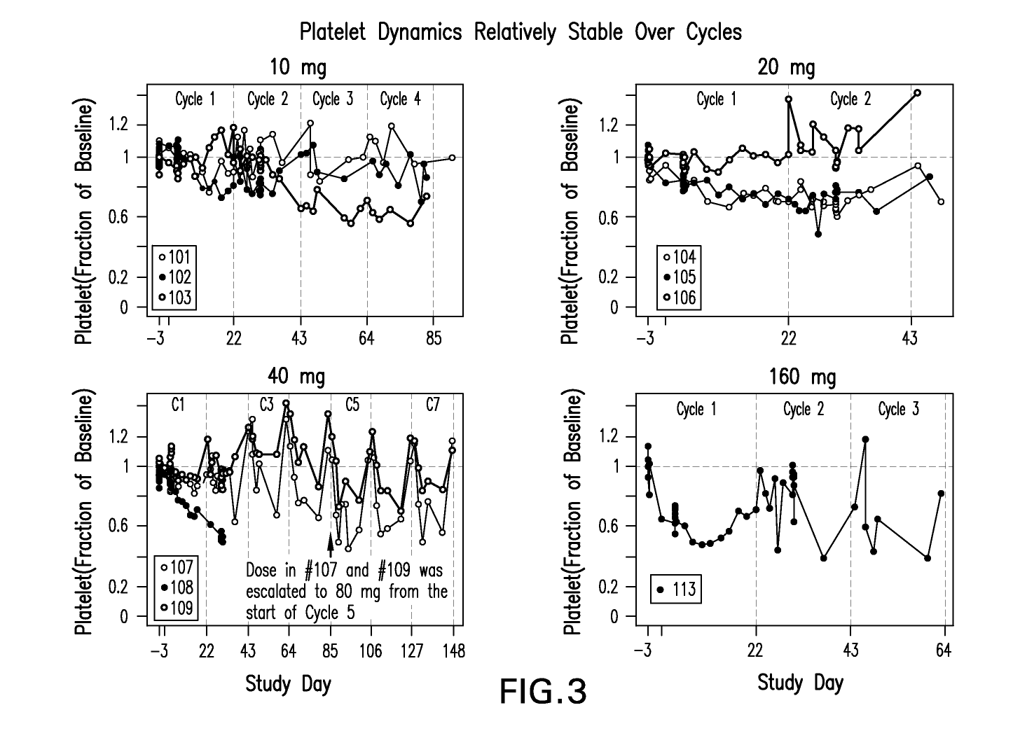

FIG. 4 illustrates the timing and dose-dependency of effect of ABT-263 on platelets.

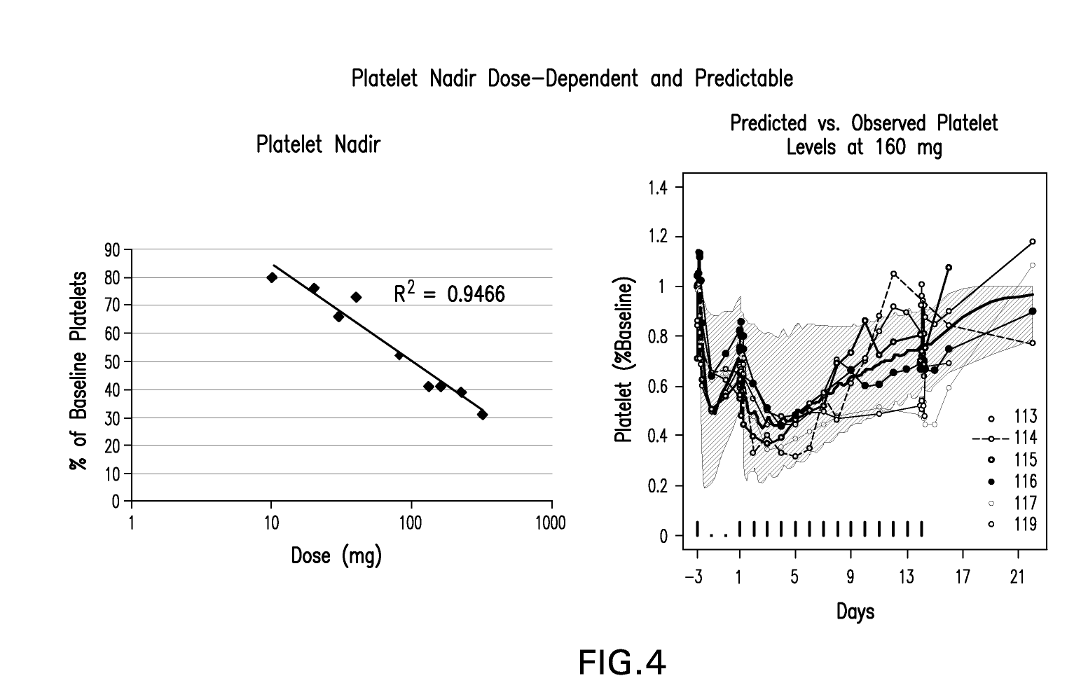

### SUMMARY OF THE INVENTION

The present invention relates to a composition for oral administration comprising N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide, Phosal® 53 Medium Chain Triglyceride and dehydrated ethanol.

### DETAILED DESCRIPTION OF THE INVENTION

The compound N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide is also referred to herein as ABT-263.

ABT-263 is a small molecule Bcl-2 family protein inhibitor that binds with high affinity (Ki≦1 nM) to multiple anti-apoptotic Bcl-2 family proteins including Bcl-XL, Bcl-2, Bcl-w, and Bcl-B. By binding to these proteins, ABT-263 releases the pro-apoptotic family members, thus resulting in cell death by apoptosis. ABT-263 displays potent mechanism-based cytotoxicity (EC₅₀≦1 μM) against human tumor cell lines derived from small cell lung carcinomas and lymphoid malignancies. ABT-263 also displays potent single agent activity against 10 of 22 cell lines consisting of multiple leukemia and lymphoma types spanning both B-cell and T-cell malignancies.

Metabolites of ABT-263, produced by in vitro or in vivo metabolic processes, may also have utility for treating cancer.

Certain precursor compounds of ABT-263 may be metabolized in vitro or in vivo to form ABT-263 and may thereby also have utility for treating cancer.

Therapeutically effective amounts of a ABT-263 depend on recipient of treatment, disease treated and severity thereof, composition comprising it, time of administration, route of administration, duration of treatment, potency, rate of clearance and whether or not another drug is co-administered.

ABT-263 may be administered with or without an excipient. Excipients include, for example, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

In two flank models of diffuse large B-cell lymphoma (DoHH-2 and WSU-DLCL2), significant monotherapy activity was noted when ABT-263 was administered at a dose of 100 mg/kg/day given p.o., q.d.×17 days. Both of these tumors are known to express high levels of Bcl-2 due to the t(14;18) translocation. The WSU-DLCL2 line was isolated from a patient whose disease progressed following chemotherapy, radiation therapy and bone marrow transplantation and is recognized as a model of therapy-resistant lymphoma.

The pharmacokinetic profile of ABT-263 was evaluated in multiple animal models including CD-1 mouse, Sprague-Dawley rat, beagle dog and cynomolgus monkey. The nonclinical pharmacokinetic profile of ABT-263 is characterized by very low plasma clearance and low volumes of distribution in all species studied, with terminal half-lives in the range of 4.6 to 8.4 hours. The oral bioavailability of the compound is formulation dependent, with values of 30% to 50% obtained from prototype solid dispersion and lipid-based formulations in dog. In rat, (¹⁴C) ABT-263 is slowly absorbed, with clearance of the metabolites primarily in the bile. Elimination of total radioactivity is rapid, with 90% of the dose recovered within 24-hours post-dose. Parent drug is the major component in systemic circulation.

Based on preclinical evidence, potential treatment-related side effects may include drug interactions, lymphopenia, testicular effects, and thrombocytopenia. At the expected biologically effective plasma concentration in humans of 6.7 .iM, ABT-263 is likely to inhibit the metabolism of drugs that are substrates for CYP2C8 and CYP2C9. Simulation of 350 mg q.d. dosing in humans describes an AUC of 92 .ig.hr/mL at steady state, with a Cₘₐₓ of ′6.5 .ig/mL. Under these conditions, platelet values are expected to be ′25 K/.iL at steady state. At the lower end of the predicted efficacious range, an AUC of 53 .ig.hr/mL (predicted 200 mg q.d. dosing in humans) is expected to be attainable while still maintaining platelet values above 50 K/.iL at steady state.

### Toxicology

The potential genetic toxicity of ABT-263 was evaluated in the Salmonella-Escherichia coli bacterial mutation assay, chromosomal aberration assay in cultured human peripheral blood lymphocytes, and in an in vivo rat micronucleus assay. ABT-263 was not mutagenic in the Ames assay, with or without metabolic activation, did not induce chromosome aberrations in human lymphocytes in vitro, with or without metabolic activation, and was not clastogenic in the in vivo rat micronucleus assay. These findings suggest that ABT-263 is not genotoxic.

In rats, ABT-263-related toxicities included mild to marked decreases in platelets, minimal to moderate decreases in lymphocytes, and minimal to mild increases in liver enzymes (alanine aminotransferase (ALT), aspartate aminotransferase (AST), and alkaline phosphatase). Although rats had mild to marked decreases in platelets following a single administration of ABT-263, there was a partial recovery of platelets during repeated administration that likely represents the normal physiologic response to decreases in platelets. Platelet levels returned to normal after cessation of ABT-263 treatment. Additionally, the ABT-263-related microscopic changes observed in rats included single-cell necrosis in multiple epithelial cell types (hepatocytes, nasal epithelium, parotid salivary gland, pancreas, seminal vesicles, and ureters), depletion of spermatogonia and spermatocytes, and a decrease in cortexical and medullary lymphocytes in the thymus consistent with thymic atrophy. Based on data from the 4-week rat toxicity study, a no-observed-adverse-effect-level (NOAEL) was not reached because of effects on platelets at all doses; however, the 10 mg/kg/day dose was considered a tolerated dose.

In dogs, toxicities included a marked decrease in platelets and a mild decrease in lymphocytes. In a 2-week toxicity study, dogs with low platelet counts (<50 K/μL) tended to have a prolonged bleeding time compared to either control dogs or their own baseline values. In a 4-week toxicity study in dogs, there was a marked decrease in circulating platelet counts that then increased during the treatment period at some doses. This effect on platelets was fully reversible in less than 7 days after ABT-263 treatment ended. Target organs in the dog included lymphoid tissues (spleen, Peyer's patch, lymph nodes, and thymus (a 2-week study)), male reproductive tissues (testes and epididymides), and pancreas. The microscopic changes observed in these target organs included decreased size of follicles, cortex, and/or germinal centers in lymphoid tissue; decreased cellularity in mantle zone, germinal centers, and/or interfollicular areas of lymphoid tissue; spermatogonia, spermatocyte and/or spermatid (round and elongated) depletion; and pancreatic acinar cell single cell necrosis. With the exception of pancreatic acinar cell single cell necrosis, these microscopic changes were also noted following a 4-week dose-free recovery period. The NOAEL following 4 weeks of administration of ABT-263 to dogs was 1 mg/kg/day.

The primary toxicological finding of a decrease in circulating platelets in mouse, rat and dog is concentration dependent and is expected to be the dose limiting toxicity for ABT-263 in humans. Thrombocytopenia was seen after administration of a single dose and is present at the time of Cₘₐₓ. After a single dose, the platelet counts return to normal values over approximately one week with an accompanying increase in mean platelet volume. Although there were marked decreases in platelets, this toxicity was tolerated for up to 28 days in both rat and dog.

### Non-Hodgkin's Lymphoma (NHL)

NHL is the sixth leading type of new cancers in the U.S. and occurs primarily in patients 60-70 years of age. NHL is a family of related diseases, which are classified on the basis of multiple characteristics including clinical attributes and histology. One method of classification places the different histologic subtypes into two major categories based on natural history of the disease: indolent and aggressive. In general, indolent subtypes grow slowly and are generally incurable whereas aggressive subtypes grow rapidly and are potentially curable. Follicular lymphomas are the most common indolent subtype, and diffuse large cell lymphomas constitute the most common aggressive subtype. The oncoprotein Bcl-2 was originally described in Non-Hodgkin's B Cell Lymphoma. Treatment of follicular lymphoma typically consists of biologically-based or combination chemotherapy. Therapy with rituximab, cyclophosphamide, doxorubicin, vincristine, and prednisone (R-CHOP) is routinely used, as is rituximab, cyclophosphamide, vincristine, and prednisone (RCVP). Single-agent rituximab (targeting the uniformly expressed CD20) and fludarabine are also used; rituximab (Rituxan) added to chemotherapy regimens has shown improved response rates and increased progression-free survival (PFS). Radioimmunotherapy agents and stem cell transplants may be used to treat refractory disease. However, since cure is rare with standard therapy, current guidelines recommend that patients be treated in the context of a clinical trial, even in first line settings. First line treatment of patients with aggressive large B-cell lymphoma typically consists of rituximab, cyclophosphamide, doxorubicin, vincristine, and prednisone (R-CHOP), or etoposide, prednisone, vincristine, cyclophosphamide, and doxorubicin (DA-EPOCH-R). High dose chemotherapy and stem cell transplant may also be used to treat relapsed or refractory disease. Currently, there is not an approved treatment regimen that produces a cure and current guidelines recommend that patients be treated in the context of a clinical trial, even in the first line setting.

Most lymphomas respond initially to any one of these therapies, but tumors typically recur and eventually become refractory. As the number of regimens patients receive increases, the more chemotherapy resistant the disease becomes. Average response to first line therapy is approximately 75%, 60% to second line, 50% to third line, and 35-40% to fourth line. Response rates with a single-agent in the multiple relapsed setting approaching 20% are considered positive and warrant further study.

Current chemotherapeutic agents elicit their antitumor response by inducing apoptosis through a variety of mechanisms. However, many tumors ultimately become resistant to these agents. Bcl-2 and Bcl-XL have been shown to confer chemotherapy resistance in both short-term survival assays in vitro and more recently, in vivo. This suggests that therapies aimed at suppressing the function of Bcl-2 and Bcl-XL might successfully overcome this chemotherapy resistance.

Lymphoid malignancies are an attractive target for ABT-263 due to Bcl-2 overexpression in a high percentage of patients. Thus, a Phase 1/2a study evaluating the safety, pharmacokinetics, and preliminary efficacy of the orally administered Bcl-2 family protein inhibitor, ABT-263, in subjects with relapsed or refractory lymphoid malignancies was initiated. The Phase 1 is the dose escalation portion of the study and the Phase 2a is the safety expansion portion of the study at the recommended Phase 2 dose.

The study consisted of two distinct portions. The Phase 1 portion of the study evaluated the pharmacokinetic profile and safety of ABT-263 in approximately 30-40 subjects following dose escalation with the objective of defining the dose limiting toxicity (DLT) and the maximum tolerated dose (MTD). The effect of food on bioavailability was also assessed in Phase 1. Subjects were enrolled at approximately eight research sites for the Phase 1 portion of the study. The Phase 2a portion of the study evaluated ABT-263 at the defined recommended Phase 2 dose (RPTD) in approximately 40 subjects with follicular lymphoma and 20 subjects with aggressive large B-cell lymphoma to obtain additional safety information and a preliminary assessment of efficacy as defined in Section. Arm A had approximately 20 subjects with relapsed or refractory follicular lymphoma. Arm B had approximately 20 subjects with follicular lymphoma that have become resistant to rituximab (progressed within 6 months of a previous rituximab treatment). Arm C had approximately 20 subjects with aggressive large B-cell lymphoma. Subjects in the Phase 2a portion of the study was enrolled at approximately twenty research sites.

### Phase 1

Inclusion Criteria

A subject was eligible for study participation if he/she: was about 18 years old; had a histologically documented diagnosis of a lymphoid malignancy as defined in the World Health Organization (WHO) classification scheme; had received at least 1 prior chemotherapy treatment regimen for a lymphoid malignancy and their disease is refractory or the subject has experienced progressive disease following the treatment; if, over the age of 70, had documented brain imaging (MRI or CT) negative for subdural or epidural hematoma within 28 days prior to the first dose of study drug; had an Eastern Cooperative Oncology Group (ECOG) performance score of about 1; if receiving selective serotonin reuptake inhibitor anti-depressants (e.g., Prozac), must have been receiving a stable dose for at least 21 days prior to the first dose of study drug; had adequate bone marrow, renal and hepatic function per local laboratory reference range as follows: bone marrow: absolute neutrophil count (ANC) of about 1,000/il; platelets of about 100,000/mm³; and hemoglobin of about 9.0 g/dL; renal function: serum creatinine of about 2.0 mg/dL or calculated creatinine clearance of about 50; hepatic function and enzymes: AST and ALT of bout 3.0×the upper normal limit (ULN) of institution's normal range; Bilirubin˜1.5×ULN. Subjects with Gilbert's Syndrome may have a Bilirubin>1.5×ULN; coagulation: aPTT, PT not to exceed 1.2×ULN. Female subjects had to be surgically sterile, postmenopausal for at least one year or have negative results for a pregnancy test and non-vasectomized male subjects must have practiced birth control.

ABT-263 Administration

For the first cycle of Phase 1, ABT-263 was administered on Day-3 (single day of dosing 3 days prior to Day 1 of Cycle 1) and Days 1 through 14 followed by seven days off drug to complete a 24-day cycle (Cycle 1 only). All subjects received ABT-263 under fasting conditions on Day-3 and under non-fasting conditions (after a standard breakfast) on Day 1 to study the effect of food on the ABT-263 pharmacokinetic profile. There was no drug administered for the 72 hours following the first dose of the first cycle (Day-3) in order to assess the single-dose pharmacokinetics and toxicity. ABT-263 was administered for 14 consecutive days followed by seven days off drug (21-day cycle) for all subsequent cycles. Except for Days-3 and 1 of the first cycle, subjects was instructed to self-administer ABT-263 orally once daily (QD) at approximately 30 minutes after breakfast on dosing days in Phase 1. The effect of food on pharmacokinetics was evaluated and changes was initiated if fasting conditions are superior.

ABT-263 Dosing

Dosing with ABT-263 began at 10 mg and escalate to MTD with a minimum of three subjects in each cohort. The study drug dose was escalated with a doubling of dose until one grade 3 or two grade 2 toxicities occurred, after which dose escalation was continued in standard 25%-40% increments. This range allows for accurate dispensing of the oral dose from the required syringe. Platelet levels after each cohort were reviewed by the investigator and the Medical Monitor and informed all dose escalation decisions. Predicted efficacious concentrations of ABT-263 were expected to occur in the range of 200 mg to 350 mg.

The first subject in each dose cohort must complete two weeks of dosing before additional subjects may be enrolled. This provision might be discontinued as safety information was reviewed from early cohorts by the investigator and the Medical Monitor and it is determined that dose escalation can proceed safely without a stagger in subject enrollment. Escalation of ABT-263 to the next dose level will proceed if all assigned subjects in a given cohort complete the cycle without experiencing a dose-limiting toxicity (DLT). If one subject within any dose level experiences a DLT, a total of six subjects was enrolled at that dose level. If ≧2 of 6 subjects experience a DLT, the previous dose was considered for MTD or dose de-escalation may occur as follows:

If two or more subjects in a cohort experiences a DLT at any dose level after doubling of the previous dose, the next dose level was reduced by 20-25% from the current dose. If less than two of six subjects experience a DLT at this reduced level, this dose was declared MTD. An additional 20-25% dose reduction may be needed if two or more subjects in a cohort still experiences a DLT. If the 20-25% dose reduction tested is deemed well tolerated (0/3 DLTs) then a 10-15% increase may be initiated at the discretion of the sponsor and the investigator(s).

MTD were defined at the highest dose level at which less than 2 of 6 subjects experience a DLT.

TABLE 2
Dose Escalation Guidelines

| Number of | |
|---|---|
| Subjects with DLT | Dose Escalation |
| 0 of 3 | Begin enrollment in the next dose level |
| 1 of 3^{a} | Enroll a total of six subjects in current dose level |
| 1 of 6^{a} | Begin enrollment in the next dose level |
| ≧2 of 6 with DLT | Previous dose determined as MTD or dose de- |

^{a}Revert to standard 25%-40% dose escalation

Subject assessments for safety and clinical progression continued weekly through the first 2 cycles. Subsequently, subject assessments for safety and clinical progression was performed once every cycle (every three weeks).

### Intra-Subject Dose Escalation and Continuous Dosing in Phase 1

To collect information on an alternative dosing schedule with ABT-263, subjects might change to a continuous dosing schedule for the 21-day cycle at their current assigned dose level. Subjects needed to complete 2 cycles at the original dosing schedule of 14 days of dosing followed by 7 days off drug. Subjects may be deemed eligible for the continuous dosing schedule if they tolerated the first 2 cycles of drug and the Medical Monitor agrees on their eligibility.

Once a subject changes to a continuous dosing schedule, the subject remained on that schedule (even if the subject later dose escalates) unless a change is warranted due to toxicity, based on the judgment of the investigator.

In addition, to maximize the collection of information at relevant doses and to minimize the exposure of individuals to sub-optimal doses, subjects may progressively escalate their current dose to the highest dose level tolerated through 2 cycles of ABT-263 administration. Individuals will need to complete at least two cycles at their originally assigned dose level, as well as subsequent dose levels, prior to any dose escalation.

All intra-subject dose escalation and continuous dosing decisions was based on the judgment of the investigator in coordination with the Abbott Medical Monitor. Once the MTD is declared and the RPTD is determined, subjects who remain on study and continue to tolerate the drug may escalate to the dose level determined to be the RPTD or the dose level below the RPTD under a continuous dosing schedule. The RPTD was defined by observed DLTs and determination of MTD.

Subject assessments for safety (physical examination, vitals signs, chemistry, hematology, urinalysis and adverse event assessment) were performed weekly during the first cycle at the new escalated dose or new dose schedule and then resumed to once every cycle. All other procedures (platelet count, echocardiogram and ECG) were performed according to the schedule of assessments.

Transition of ABT-263 Dosing from Phase 1 Part to Phase 2a Part

Once the MTD is reached, enrollment into the Phase 1 portion of the study ended and a safety analysis was performed. The results of the safety analysis as well as the recommended Phase 2 dose was communicated to all participating research sites prior to the start of enrollment in the Phase 2a portion of the study. Phase 1 subjects are not eligible for enrollment in the Phase 2a portion of the study but may continue receiving ABT-263 for up to one year provided they continue to tolerate the drug, have no evidence of disease progression, and do not meet any of the criteria for subject discontinuation.

### Phase 2a

Inclusion Criteria

A subject was eligible for study participation if he/she: was ≧18 years of age; had a histologically documented diagnosis of follicular lymphoma (subjects enrolling in Arm C must had a histologically documented diagnosis of aggressive large B-cell lymphoma); had a measurable disease by International Working Group (IWG) Criteria for Tumor Response; had met one the following criteria: follicular lymphoma (Arm A) having received at least one and no more than four prior conventional chemotherapy regimens and their disease was refractory or the subject had experienced progressive disease following treatment; follicular lymphoma (Arm B) that had become resistant to rituximab (i.e. progressed within 6 months of a previous rituximab treatment); aggressive large B-cell lymphoma (Arm C) after having received at least one and no more than four prior conventional chemotherapy regimens and their disease is refractory or the subject has experienced progressive disease following the treatment; if clinically indicated (e.g., subjects over the age of 70), had documented brain imaging (MRI or CT) negative for subdural or epidural hematoma within 28 days prior to the first dose of study drug; had archived diagnostic tissue available for assessment of Bcl-2 family protein expression; had one of the following available for pharmacodynamic analyses:core needle biopsy of malignant lymph node obtained at screening; bone marrow aspirate or core obtained at screening, positive for lymphoma; archived tumor tissue with no intervening treatment since the biopsy (e.g., from debulking, tissue obtained at relapse or bone marrow sample); had an Eastern Cooperative Oncology Group performance score of about 1; for subjects receiving Selective Serotonin Reuptake Inhibitor (SSRI) anti-depressants (e.g., Prozac) must have had a stable dose for at least 21 days prior to the first dose of study drug; had adequate bone marrow, renal and hepatic function per local laboratory reference range as follows: bone marrow: absolute neutrophil count (ANC) of about 1,000/il; platelets of about 100,000/mm³; hemoglobin of about 9.0 g/dL; renal function: serum creatinine of about 2.0 mg/dL or calculated creatinine clearance of about 50; hepatic function and enzymes: AST and ALT of about 3.0× the upper normal limit (ULN) of institution's normal range; bilirubin of about 1.5×ULN. Subjects with Gilbert's Syndrome may have a Bilirubin>1.5×ULN; coagulation: aPTT, PT not to exceed 1.2×ULN. Female subjects had to be surgically sterile, postmenopausal for at least one year or have negative results for a pregnancy test and non-vasectomized male subjects must have practiced birth control.

### ABT-263 Dosing

ABT-263 was administered for 14 consecutive days followed by 7 days off drug for the Phase 2a portion of the study. Arm A will evaluate ABT-263 in subjects with relapsed or refractory follicular lymphoma, Arm B will evaluate ABT-263 in subjects with rituximab resistant follicular lymphoma, and Arm C will evaluate ABT-263 in subjects with aggressive large B-cell lymphoma. All subjects will self-administer ABT-263 at approximately 30 minutes after breakfast, unless results from the Phase 1 food effect study indicate fasting conditions are superior. If, during Phase 2a, dose-limiting toxicities were observed at a frequency higher than the definition of MTD (>33%), the investigator and reviewed the data and determined whether dosing should continue or a new, lower recommended Phase 2 dose was defined.

### Physical Examination

A physical examination (including weight) were performed at Screening, Cycle 1 Day-3 (Phase 1) or Cycle 1 Day 1 (Phase 2a), Day 1 of each subsequent cycle (or within 72 hours prior), at Final Visit and at the Safety Follow-up Visit. A symptom-directed physical examination were performed weekly through the first 2 cycles and when necessary. Height were measured only at Screening. The physical examination performed at Screening will serve as the baseline physical examination for clinical assessment. Any clinically significant physical examination findings after dosing were recorded as adverse events.

### Vital Signs

Body temperature (oral), blood pressure and pulse were measured at Screening, Cycle 1 Day-3 (Phase 1) or Cycle 1 Day 1 (Phase 2a), weekly through the first 2 cycles, Day 1 of each subsequent cycle (or within 72 hours prior), at Final Visit and at the Safety Follow-up Visit. The vital sign measurements at Screening will serve as the baseline measurements for clinical assessment.

Blood pressure and pulse rate were measured 30 to 60 minutes after study drug administration and after the subject has been sitting for at least 5 minutes.

### Platelet Count

Platelet counts were performed stat and assessed by the investigator or subinvestigator prior to study drug administration as follows:

### Phase 1:

Screening

Cycle 1:
Day-3 through Day 2
   Platelet counts were drawn at all of the pharmacokinetic sampling time points on Days-3 through 2.
Days 3, 4, 5, 6 and 8
   If platelet count is below 50,000/mm³ on Day 8, platelet counts were drawn daily on Day 9 and Day 10.
Day 11
   If platelet count is below 50,000/mm³ on Day 11, platelet counts were drawn daily on Day 12 and Day 13.
Day 14
   Platelet counts were drawn at all of the pharmacokinetic sampling timepoints on Day 14, as indicated in Table 5.
Day 16
As needed

Subsequent Cycles:
Day 1, 3, 5, 8 and 16
As needed

Final Visit and Safety Follow-up Visit

### 21/21 Day Continuous Dosing Schedule-Phase 1b

### Screening

### Lead In Period

Lead-In Days 1, 2, 3, 5 and 7

If the lead-in period extends beyond 7 days, platelet counts will be drawn prior to dosing on each additional lead-in day (Lead-in Days 8-14).

### Cycle 1:

Day 1
   Platelet counts will be drawn at all of the pharmacokinetic sampling time points on Day 1
Days 2, 3, 5 and 8
Day 14
   Platelet counts will be drawn at all of the pharmacokinetic sampling time points on Day 1
Day 16
As needed

### Subsequent Cycles:

Weekly
   If a platelet count on any given day is less than 50,000/mm3, additional platelet counts should be performed every day or at the discretion of the investigator.
   As needed

### Final Visit and Safety Follow-up Visit

### Phase 2a:

Screening Cycle 1:
Days 1, 2, 3, 5, 8, 14 and 16
As needed

Subsequent Cycles
Weekly and as needed
If the investigator and Abbott Medical Monitor jointly agree that platelet counts through Cycle 4 have been stable, then the frequency of platelet counts in Cycle 5 and beyond may be decreased to Day 1 of each Cycle and as needed.

Final Visit and Safety Follow-up Visit

Phase 1a, Phase 1b and Phase 2a
If a platelet count on any given day is less than 50,000/mm3, additional platelet counts should be performed very day or at the discretion of the investigator.
If a platelet transfusion is necessary, a post-transfusion platelet count should be obtained within 10-60 minutes of the transfusion being completed.

The platelet count measurement obtained on Cycle 1 Day-3 (pre-dose) will serve as the baseline for clinical assessment in the Phase 1a portion of the study.

The platelet count measurement obtained on Lead-In Day 1 (pre-dose) will serve as the baseline for clinical assessment in the Phase 1b portion of the study.

The platelet count measurement obtained on Cycle 1 Day 1 (pre-dose) will serve as the baseline for clinical assessment in the Phase 2a portion of the study.

Platelet count from an automated reading is less than 25,000/mm³ should be confirmed the same day by manual reading and a separate peripheral draw. Additional platelet counts will be obtained from a subject if ABT-263 is either held or interrupted per the management guidelines.

All platelet count measurements obtained through Cycle 4 in Phase 1a and Phase 1b will be faxed to the Oncology Group Safety Desk within 24 hours via the contact information provided in Section 6.5.

The platelet count schedule of assessment may be modified as information is obtained regarding the expected decrease in platelets in response to study drug administration. This will be based upon discussion between the investigator and the Abbott Medical Monitor.

The lymphocyte enumeration results from Screening will serve as the baseline for clinical assessment.

Lymphocyte enumeration to identify B and T cell lymphocyte subpopulations were performed at Screening, Cycle 1 Day 14, after Cycle 4 and at the Final Visit for each subject. All lymphocyte enumeration results obtained in Phase 1a and Phase 1b portions of the study will be faxed to the Oncology Group Safety Desk as soon as they are available via the contact information provided in Section 6.5.

### Status

The ECOG performance status were assessed at Screening, Cycle 1 Day-3 (Phase 1a) or Cycle 1 Day 1 (Phase 1b and Phase 2a), weekly through the first two cycles, Day 1 of each subsequent cycle (or within 72 hours prior), at the Final Visit and at the Safety Follow-up Visit as follows:

| Grade | Description |
|---|---|
| 0 | Fully active, able to carry on all pre-disease performance without |
| | restriction. |
| 1 | Restricted in physically strenuous activity but ambulatory and |
| | able to carry out work of a light or sedentary nature, e.g., |
| | light housework, office work. |
| 2 | Ambulatory and capable of all self-care but unable to carry out |
| | any work activities. Up and about more than 50% of waking |
| | hours. |
| 3 | Capable of only limited self care, confined to bed or chair |
| | more than 50% of waking hours. |
| 4 | Completely disabled. Cannot carry on any self-care. Totally |
| | confined to bed or chair. |

An ECOG performance status will also be assessed on Lead-in Day 1 (Phase 1b). The ECOG performance status assessed at Screening will serve as the baseline for clinical assessment.

### 12-Lead Electrocardiogram (ECG) (Phase 1)

A 12-lead resting ECG were performed for all subjects in the first 2 cohorts at Screening, Cycle 1 Day-3, Cycle 1 Day 14, Day 1 of each subsequent cycle and at the Final Visit. For subsequent cohorts, an ECG were performed at Screening, Cycle 1 Day-3 and Day 14, Cycle 3 Day 1 and Day 14 and at the Final Visit.

For subjects enrolled in Phase 1b, Eggs will be performed at Screening, Cycle 1 Day 1, Cycle 1 Day 14, Cycle 3 Day 1, Cycle 3 Day 14 and Final Visit. An ECG will also be performed on Lead-in Day 1 during the lead-in period. The data were assessed on an ongoing basis and ECG monitoring may be adjusted depending on the observation of any clinically significant findings.

All ECGs should be taken approximately 6-8 hours post-dose (2-8 hours post-dose is an acceptable timeframe), and if possible at approximately the same time of day. If pharmacokinetic data indicates the Cₘₐₓ of parent drug or a major metabolite occurs at a time different than this specified range, the timing of the ECG were modified. A qualified physician will sign and date the ECGs, determine if any findings outside normal physiological variation are clinically significant (in consultation with a cardiologist, if necessary) and document this on the appropriate CRF. The original ECG tracing with physician assessment were retained in the subject's records at the study site and a copy were faxed to the Oncology Group Safety Desk via the contact information. The ECG measurement obtained at Screening were used to document baseline status of the subject so that safety comparisons can be made, if necessary. Repeat ECGs were performed whenever clinically necessary.

### 12-Lead Electrocardiogram (ECG) (Phase 2a)

A 12-lead resting ECG were performed for all subjects in the Phase 2a portion of the study at Screening, on Cycle 1 Day 14, Cycle 3 Day 14 and at the Final Visit. All ECGs should be taken approximately 6-8 hours post-dose (2-8 hours post-dose is an acceptable timeframe), and at approximately the same time of day. If pharmacokinetic data indicates the Cₘₐₓ^{of} parent drug or a major metabolite occurs at a time different than this specified range, the timing of the ECG may be modified. A qualified physician will sign and date the ECGs, determine if any findings outside normal physiological variation are clinically significant (in consultation with a cardiologist, if necessary) and document this on the appropriate CRF. The original ECG tracing were retained in the subject's records at the study site and a copy will be faxed to the Oncology Group Safety Desk via the contact information provided in Section 6.5. The ECG measurement obtained at Screening were used to document baseline status of the subject so that safety comparisons can be made, if necessary. Repeat ECGs were performed whenever clinically necessary.

2D Echocardiogram with Doppler (Phase 1a and Phase 1b)

In Phase 1a, a 2D echocardiogram with Doppler were performed for all subjects in the first 2 cohorts at Screening, Cycle 1 Day-3 and Day 14, Day 1 of each subsequent cycle and at the Final Visit. For subsequent cohorts in Phase 1a, an echocardiogram with Doppler were performed at Screening, Cycle 1 Day-3 and Day 14, Cycle 3 Day 1 and Day 14 and at the Final Visit. For subjects enrolled in Phase 1b, an echocardiogram with Doppler will be performed at Screening, Cycle 1 Day 1, Cycle 1 Day 14, Cycle 3 Day 1 Cycle 3 Day 14 and Final Visit. An echocardiogram will be performed on Lead-in Day 1 during the lead-in period.

If necessary, the Cycle 1 Day 14 and Cycle 3 Day 14 echocardiograms may be performed within 3 days prior to Day 14. An echocardiogram will also be performed on Lead-in Day 1 during the lead-in period. The test results were assessed on an ongoing basis and monitoring may be adjusted depending on the observation of any clinically significant findings.

All echocardiograms should be taken approximately 6-8 hours post-dose (2-8 hours post-dose is an acceptable timeframe), and if possible at approximately the same time of day. If pharmacokinetic data indicates the Cₘₐₓ of parent drug or a major metabolite occurs at a time different than this specified range, the timing of the echocardiogram were modified. A qualified physician will sign and date the echocardiogram reports, determine if any findings outside normal physiological variation are clinically significant and document this on the appropriate CRF. The original echocardiogram report with physician assessment were retained in the subject's records at the study site and a copy were faxed to the Oncology Group Safety Desk via the contact information provided in Section 6.5. In addition, Abbott will require access to the recording of the echocardiogram as necessary. The echocardiogram results obtained at Screening were used to document baseline status of the subject so that safety comparisons can be made, if necessary. Repeat echocardiograms were performed whenever clinically necessary.

### Bone Marrow Biopsy

A bone marrow biopsy were performed for all subjects at Screening (within 21 days prior to the first does of study drug) to determine disease involvement in the marrow and for pharmacodynamic analysis.

Bone marrow biopsy for pharmacodynamic analysis is described in Section 5.3.7. The bone marrow biopsy at Screening should be performed after all other eligibility criteria have been met, unless otherwise obtained through standard of care. For subjects with bone marrow involvement at study entry, a repeat bone marrow biopsy should be obtained if the subject's best response to ABT-263 is determined to be a Complete Response (CR). This should be performed within 8-12 weeks after criteria (CR) are first met.

### Bone Marrow Aspirate and Biopsy for NCI-WG Criteria

A bone marrow biopsy will be performed at Screening (within 21 days prior to the first dose of study drug) unless a bone marrow aspirate and biopsy was obtained within 12 weeks of staring study drug without intervening treatment and is representative of the subject's existing CLL. The bone marrow aspirate and biopsy should be performed after all eligibility criteria have been met, unless otherwise obtained through standard of care.

If a subject meets all the clinical and laboratory criteria for a complete response (CR)(except for platelet counts due to potential drug related toxicity), a bone marrow aspirate and biopsy should be performed 3 months after the criteria are first met in order to confirm a CR.

Bone marrow aspirates and biopsies performed as standard of care throughout the study should also be captured on a case report form.

### Tumor Assessments for IWG Criteria

Computed Tomography (CT) of involved anatomic regions, magnetic resonance imaging (MRI, if medically indicated) and bone marrow biopsy (if medically indicated) were used in evaluation of all subjects using the IWG criteria for tumor response at Screening, at the end of Cycle 2 and Cycle 4, every 3^{rd} cycle thereafter and at the Final Visit. Subjects will continue to be monitored by the same methods unless evidence of tumor metastasis warrants otherwise. The tumor assessment performed at Screening will serve as the baseline for clinical assessment. Response criteria definitions are outlined in Section 5.33.1.

A PET scan using fluorodeoxyglucose (FDG) will be used to differentiate between an unconfirmed complete response (Cru) and complete response (CR) for subjects enrolled in Phase 2a with diffuse large B-cell lymphoma (Arm C). The following IWG criteria for complete response will be used for this population:
Typically FDG-avid lymphoma: in patients with no pretreatment PET scan or when the PET scan positive before therapy a post-treatment residual mass of any size is permitted as long as it is PET negative.
Variably FDG-avid lymphomas/FDG avidity unknown: in patients without a pretreatment PET scan, or if a pretreatment PET scan was negative, all lymph nodes and nodal masses must have regressed on CT to normal size (less than or equal to 1.5 cm their greatest diameter for nodes>1.5 cm before therapy). Previously involved nodes that were 1.1-1.5 cm in their long axis and more than 1.0 cm in their short axis before treatment must have decreased to ≦1.0 cm in their short axis after treatment.

Subjects will continue to be monitored by the same methods unless evidence of tumor metastasis warrants otherwise. The tumor assessment performed at Screening will serve as the baseline for clinical assessment.

### Tumor Assessments for NCI-WG Criteria

Analysis of peripheral blood, physical examination, bone marrow aspirate and biopsy, CT scan of involved anatomical regions and MRI (if medically indicated) will be used.

Subjects will be evaluated against the NCI-WG criteria₂₁ (physical examination/CT/MRI) at the end of Cycle 2, the end of Cycle 4, the end of every 3rd cycle thereafter, and at the Final Visit. Analysis of peripheral blood will be evaluated against the NCI-WG criteria for tumor response assessment on Day 1 (pre-dose) of the following cycle. For example, when a subject completes Cycle 2, the laboratory values from Day 1 of Cycle 3 (pre-dose) will be used to assess tumor response.

A CT scan of involved anatomic regions (or MRI, if medically indicated) will be done at Screening (within 21 days prior to the first dose of study drug). The tumor assessment performed at Screening will serve as the baseline for clinical assessment.

If a subject meets all the clinical and laboratory criteria for a complete response (CR) or a partial response (PR) (except for platelet counts due to potential drug related toxicity), a CT scan should be performed 3 months or 2 months respectively, after the criteria are first met in order to confirm a CR or PR.

CT scans and MRIs performed as standard of care throughout the study should also be captured on a case report form.

### Pregnancy Test

For female subjects of childbearing potential, the local reference laboratory will perform a serum pregnancy test at Screening and a urine pregnancy test before dosing on Cycle 1 Day-3 (Phase 1) or Cycle 1 Day 1 (Phase 2a). The test results must be reviewed and determined to be negative prior to dosing.

Subjects considered not of childbearing potential must be documented as being surgically sterile or post-menopausal (for at least one year).

### Clinical Laboratory Tests

In Phase 1a and Phase 1b, local laboratories will be utilized to process and provide results for the clinical laboratory tests. The principal investigator or subinvestigator will review, initial and date all laboratory results. The laboratory test results will be collected on the Case Report Forms.

All laboratory measurements obtained through Day 1 of Cycle 2 in Phase 1a and Phase 1b, will be faxed to the Oncology Group Safety Desk within 24 hours via the contact information provided in Section 6.5.

### Phase 1a

Hematology, chemistry, and urinalysis will be collected at:
Screening
Cycle 1 Day-3
Cycle 1 Day 1, Day 2 and Day 3 (chemistry and hematology only)
Weekly through Cycle 2
Day 1 of subsequent Cycles (or within 72 hours)
Final Visit
Safety Follow-up Visit Hematology, chemistry and urinalysis samples were collected at Screening, Cycle 1 Day-3 (Phase 1) or Cycle 1 Day 1 (Phase 2a), Cycle 1 Day 2 (Phase 1) or Cycle 1 Day 3 (Phase 2a) (chemistry and hematology only), weekly through the first 2 cycles, Day 1 of each subsequent cycle (or within 72 hours prior), at Final Visit and at the Safety Follow-up Visit. Results must be available and reviewed prior to dosing. The laboratory test results from Screening (except platelet count) will serve as the baseline for clinical assessment.
   Chemistry tests should be obtained under fasting conditions if possible.
   Triglycerides will only be collected at Screening and at the Final Visit.
   Local laboratories were utilized to process and provide results for the clinical laboratory tests. The principal investigator or subinvestigator will review, initial and date all laboratory results. The laboratory test results were collected on the Case Report Forms.

TABLE 6
Clinical Laboratory Tests

| Hematology | Clinical Chemistry | Urinalysis |
|---|---|---|
| Hematocrit | Blood urea nitrogen (BUN) | Specific gravity |
| Hemoglobin | Creatinine | Ketones |
| Red blood cell (RBC) count | Total bilirubin | pH |
| White blood cell (WBC) | Serum glutamic-pyruvic | Protein |
| count | transaminase (SGPT/ALT) | Blood |
| Neutrophils | Serum glutamic-oxaloacetic | Glucose |
| Bands | transaminase (SGOT/AST) | Microscopic |
| Lymphocytes | Alkaline phosphatase | examination |
| Monocytes | Sodium | (as indicated) |
| Basophils | Potassium | |
| Eosinophils | Calcium | |
| Platelet count (estimate not | Inorganic phosphorus | |
| acceptable) | Uric acid | |
| Prothrombin time (PT) | Cholesterol | |
| Activated partial | Total protein | |
| thromboplastin | Glucose | |
| time (aPTT) | Triglycerides (Screening | |
| Mean platelet volume | and Final Visit) | |
| (MPV) | Albumin | |
| Mean corpuscular | Lactate dehydrogenase | |
| hemoglobin | (LDH) | |
| (MCH) | Magnesium | |
| Mean corpuscular volume | Chloride | |
| (MCV) | Bicarbonate | |
| Mean corpuscular | Amylase (Screening, | |
| hemoglobin | C1D14 and Final Visit) | |
| concentration (MCHC) | Lipase (Screening, C1D14 | |
| Reticulocyte count | and Final Visit) | |

For any laboratory test value outside the reference range that the investigator considers to be clinically significant:
The investigator may repeat the test to verify the out-of-range value.
   The investigator will follow the out-of-range value to a satisfactory clinical resolution.
   A laboratory test value that requires a subject to be discontinued from the study were recorded as an adverse event.

### Phase 2a

Clinical laboratory samples obtained in Phase 2a will be assessed using a certified central laboratory (Quest Diagnostics). This data will be used for all data analysis. The central laboratory for this study will provide instructions regarding the collection, processing and shipping of these samples. All laboratory samples should be shipped to the central laboratory.

### Phase 2a

Clinical laboratory samples obtained in Phase 2a will be assessed using a certified central laboratory (Quest Diagnostics). This data will be used for all data analysis. The central laboratory for this study will provide instructions regarding the collection, processing and shipping of these samples. All laboratory samples should be shipped to the central laboratory.

Hematology, chemistry, and urinalysis will be collected at:
Screening
Cycle 1 Day 1
Cycle 1 Day 2 and Day 3 (chemistry and hematology only)
Weekly through Cycle 2
Day 1 of subsequent Cycles (or within 72 hours)
Final Visit
Safety Follow-up Visit

For subjects who are at high risk for tumor lysis syndrome during Cycle 2 and beyond, additional hematology and chemistry samples may be collected as per the management guidelines in Section 6.7.5.

A certified local reference laboratory may perform hematology and chemistry tests for immediate subject management; however split or concurrent samples must be drawn and sent to the central laboratory for analysis.

Local laboratories will be utilized to process and provide results for all platelet counts. Platelet count results must be available and reviewed prior to dosing. The laboratory test results from Screening (except platelet count) will serve as the baseline for clinical assessment.

TABLE 9
Clinical Laboratory Tests

Hematology
Hematocrit
Hemoglobin
Red blood cell (RBC) count
White blood cell (WBC) count
Neutrophils
Bands
Lymphocytes
Monocytes
Basophils
Eosinophils
Platelet count (estimate not
acceptable)
Prothrombin time (PT)
Activated partial thromboplastin
time (aPTT)
Mean platelet volume (MPV)
Mean corpuscular hemoglobin
(MCH)
Mean corpuscular volume (MCV)
Mean corpuscular hemoglobin
concentration (MCHC)
Reticulocyte count
Clinical chemistry (a, b)
Blood urea nitrogen (BUN)
Creatinine
Total bilirubin
Serum glutamic-pyruvic
transaminase (SGPT/ALT)
Serum glutamic-oxaloacetic
transaminase (SGOT/AST)
Alkaline phosphatase
Sodium
Potassium
Calcium
Inorganic phosphorus
Uric acid
Cholesterol
Total protein
Glucose
Triglycerides
Albumin
Lactate dehydrogenase (LDH)
Magnesium
Chloride
Bicarbonate
Amylase (Screening,
C1D14 and Final Visit)
Lipase (Screening, C1D14
and Final Visit)
Urinalysis
Specific gravity
Ketones
pH
Protein
Blood
Glucose
Microscopic examination
(as indicated)
a. Chemistry tests should be obtained under fasting conditions if possible.
b. Triglycerides will only be performed at Screening and Final Visit.

For any laboratory test value outside the reference range that the investigator considers to be clinically significant:
The investigator may repeat the test to verify the out-of-range value.
The investigator will follow the out-of-range value to a satisfactory clinical resolution.
A laboratory test value that requires a subject to be discontinued from the study will be recorded as an adverse event.

### Assignment of Subject Numbers

For the Phase 1 portion of the study, the results of all screening and pre-dose Study Day 1 (or Lead-in Day 1 for lead-in period) evaluations must be within clinically acceptable limits, upon review by the investigator with the concurrence of the Abbott Medical Monitor or designee, before a subject can be administered study drug. Subjects will not be enrolled in the study if laboratory or other screening results are not within clinically acceptable limits. Subjects who meet the inclusion criteria and do not meet any of the exclusion criteria were assigned a unique subject number.

The results of all screening and pre-dose Study Day 1 evaluations must be within clinically acceptable limits (per inclusion criteria in Section 5.2.1), upon review by the investigator before a subject can be administered study drug. Subjects will not be enrolled in the study if laboratory or other screening results are not within clinically acceptable limits. Subjects who meet the inclusion criteria and do not meet any of the exclusion criteria will be assigned a unique subject number by Abbott, as described in Section 5.5.3.

### Meals and Dietary Requirements

For the first cycle in the Phase 1 study, ABT-263 were administered to all subjects under fasting conditions on Day-3 and non-fasting conditions on Days 1 through 14. Subjects may not consume grapefruit or grapefruit products within the 3-day period prior to initial drug administration and until the last treatment cycle is completed due to possible CYP3A-mediated metabolic interaction. On Day-3, subjects will not be allowed to take food or beverage, except for water to quench thirst, from 8 hours prior to dosing until after the collection of the 4-hour blood sample. No fluids except those required for dosing were allowed for 1 hour before dosing and 1 hour after dosing. On Days 1 and 14, all subjects will have a standard breakfast at study sites prior to administration of ABT-263. The meal content will consist of approximately 520 Kcal; with approximately 30% calories from fat.

### Blood Samples for Pharmacogenetic Analyses

### DNA

One 4 mL whole blood sample for DNA isolation were collected at Screening from each subject who consents to provide samples for pharmacogenetic analysis.

The samples were collected into EDTA tubes and stored under frozen conditions until shipment on dry ice to Abbott.

If pharmacogenetic testing is performed, results from individual subjects were kept coded and confidential. Abbott will store the DNA samples in a secure storage space with adequate measures to protect confidentiality. Samples were coded so that subject identities will not be available to the scientists conducting the genotyping analysis.

Specimens for Pharmacodynamic Analyses

### Blood Collection for Proteomics

Approximately 6 mL of blood were collected by venipuncture into one 6-mL EDTA (purple cap) tube at Screening, Cycle 1 Day 14, Cycle 2 Day 14, and at the Final Visit from all subjects. The collection should be performed as described below. The complete process of centrifugation, transfer to cryovial and freezing should be accomplished in less than 1 hour from blood draw.
Collect the blood samples into a 6-mL EDTA (purple top) tube.
   Immediately invert the collection tube 8-10 times (so clot formation is reduced).
   Centrifuge sample at 1200-1500×g for 15 minutes at 2-8° C.
   Within 15 minutes, transfer the plasma into a separate 4 mL labeled cryovial and freeze at −70° C.
Store samples at −70° C. until shipped to Abbott on dry ice sufficient for three days.

### Specimens for Pharmacodynamic Analyses

### Phase 1a and Phase 1b Pharmacodynamic Collections

### Mandatory Collections

Proteomics
Bone marrow aspirate for tumor cells. If a bone marrow aspirate is not collected, a bone marrow core biopsy may be obtained

### Optional Collections

DNA sample
Formalin fixed, paraffin embedded archived diagnostic tissue for IHC and FISH analysis
Core needle biopsies (two are preferred)
One core needle biopsy that is formalin-fixed for IHC and FISH analysis
One core needle biopsy that is flash frozen for CGH/microarray analysis

### Phase 2a Pharmacodynamic Collections

### Mandatory Collections

One of the following is required for all subjects in the Phase 2a portion of the study:
Core needle biopsy of malignant lymph node.
Bone marrow aspirate or core positive for lymphoma.
Archived tumor tissue with no intervening treatment since the biopsy (e.g., from debulking, tissue obtained at relapse or bone marrow sample).
Proteomics

### Optional Collections

DNA sample

If none of the above is attainable for a potential subject (Phase 1a, Phase 1b, and Phase 2a), the Abbott Medical Monitor should be contacted.

Needle biopsies will also be obtained at time of relapse from all subjects in the Phase 2a portion of the study.

### Processing of Pharmacodynamic Specimens

### Blood Collection for Proteomics

Approximately 6 mL of blood will be collected by venipuncture into one 6-mL EDTA (purple cap) tube at Screening, Cycle 1 Day 14, Cycle 2 Day 14, and at the Final Visit from all subjects. The collection should be performed as described below. The complete process of centrifugation, transfer to cryovial and freezing should be accomplished in less than 1 hour from blood draw.
Collect the blood samples into a 6-mL EDTA (purple top) tube.
Immediately invert the collection tube 8-10 times (so clot formation is reduced).
Centrifuge sample at 1200-1500×g for 15 minutes at 2-8° C.
Within 15 minutes, transfer the plasma into a separate 4 mL labeled cryovial and freeze at −70° C.
Store samples at −70° C. until shipped to Abbott on dry ice sufficient for three days.

### Tissue Collection for IHC and FISH Fixed Samples

Immunohistochemistry (IHC) and fluorescence in situ hybridization (FISH) were performed on tissue slides from archived, diagnostic, formalin fixed, paraffin embedded (FFPE) tissue blocks from all subjects who consent in the Phase 1 portion of the study and from all subjects in the Phase 2a portion of the study.

From each representative FFPE block, the local pathology laboratory should prepare 15 slices of tissue with a thickness of approximately 4-6 microns and apply to positively charged slides to be used for IHC and FISH analysis. Therefore, a minimum of (15) 4-6 microns of tissue sections should be collected from each subject block. In cases where there is not enough appropriate tissue available to provide these sections, the investigator will communicate with the pathology laboratory to determine the maximum number of slides that can be provided, and relay that information to Abbott prior to slide preparation.

To ensure optimal sampling, two quality control slides must also be prepared by the pathology laboratory and included in the shipment of slides to Abbott. These quality control slides were representative of the beginning and of the end of the tissue section. These slides are to be stained using Hematoxylin and Eosin (H & E) and reviewed by the local pathologist to ensure the diagnostic quality of viable tumor and normal cells (i.e., large regions of necrosis or areas composed primarily of fibrous connective tissue or adipose tissue are not the predominant feature). The remaining tissue prepared for the unstained slides were procured from the sections closest to the section that is of adequate diagnostic quality.

Included with each shipment should be a copy of the pathology report and completed shipment inventory form. Tissue slides were shipped in slide boxes. Slide boxes should be packaged using suitable shipping materials and sent to Abbott at ambient temperature.

### Needle Biopsies Phase 1

Needle biopsies were obtained prior to therapy and at time of relapse, when feasible, for all subjects in the Phase 1 portion of the study who consent and who have readily accessible tumor tissue. Biopsies were performed after consent, prior to drug administration and after the subject has relapsed on therapy.

### Phase 2a

One of the following is required for all subjects in the Phase 2a portion of the study:
Core needle biopsy of malignant lymph node obtained at Screening.
Bone marrow aspirate or core obtained at Screening, positive for lymphoma.
Archived tumor tissue with no intervening treatment since the biopsy (e.g., from debulking, tissue obtained at relapse or bone marrow sample).

If none of the above is attainable for a potential subject, the Abbott Medical Monitor should be contacted.

Needle biopsies will also be obtained at time of relapse from all subjects in the Phase 2a portion of the study.

It is preferred that at least two core biopsies be obtained. These biopsies should be at least 18 gauge in diameter and at least 1 cm in length. It is estimated that there were between 2-5 million cells from each biopsy. The biopsies may be processed according to the institutional standard procedures or per the following instructions. If a procedure other than what is described below is used, a description of the procedure should be provided to Abbott.

One core biopsy is to be fixed in formalin for between 8-24 hours then embedded in paraffin and stored at −20° C. until shipment to Abbott at ambient temperature. The second core biopsy specimen should be placed into properly labeled cryovial. The tumor sample were flash frozen in liquid nitrogen immediately after collection. The specimen were stored frozen at −70° C. until shipment to Abbott. Samples should be shipped to Abbott on dry ice sufficient for 3 days.

### Bone Marrow Collection Bone Marrow Aspirate

Bone marrow aspirates should be drawn at baseline in conjunction with the diagnostic biopsy. The aspirate may be processed according to the institutional standard procedures or per the following instructions. If a procedure other than what is described below is used, a description of the procedure should be provided to Abbott.

Fresh fixative solution should be prepared by adding 8 mL of concentrated fixative in tube A to tube B, which contains 32 mL of diluent (both tubes were provided and wrapped in foil), and mixed 5-6 times. Approximately 2 mL of bone marrow aspirate should be diluted in 2 mL of phosphate buffered saline (PBS), then pipetted up and down (titurated) 5-6 times with a 10 mL pipette to create a single cell suspension. This 4 mL suspension should be added to the prepared fixative, mixed 5-6 times and the samples should be placed in a −70° C. freezer until shipment to Abbott.

### Bone Marrow Core Biopsy

If bone marrow aspirate is not collected, a bone marrow core biopsy may be obtained. The core may be processed according to the institutional standard procedures or per the following instructions. If a procedure other than what is described below is used, a description of the procedure should be provided to Abbott.

The core should be fixed in freshly prepared 4% paraformaldehyde. A 10 mL ampule of 20% paraformaldehyde and a 50 mL conical tube (tube C) containing 40 mLs of 1×PBS were provided. The paraformaldehyde from the ampule should be added to tube C, mixed 4-5 times and then the core bone marrow biopsy should be added. The sample should be allowed to fix for 16-24 hours at 4° C. and then embedded in paraffin.

TABLE 7
Phase 1 Schedule of Biomarker Sample Collection

| | | Cycle 1 Day | Cycle 2 Day | |
|---|---|---|---|---|
| | Screening | 14 | 14 | Off Treatment |
| DNA | 4 mL blood^{b} | | | |
| Proteomics | 6 mL blood | 6 mL blood | 6 mL blood | 6 mL blood |
| CGH/microarray | Core needle biopsy-flash | | | Core needle |
| | frozen^{b} | | | biopsy-flash |
| | | | | frozen (time of |
| | | | | relapse)^{b} |
| IHC/FISH | Archived diagnostic | | | Core needle |
| | FFPE tissue^{b} | | | biopsy formalin- |
| | Core needle biopsy | | | fixed (time of |
| | | | | relapse)^{b} |
| Tumor Cells^{a} | 2 mL bone marrow | | | |
| | aspirate | | | |

^{a}Bone marrow aspirate samples for analysis should be obtained with the bone marrow biopsies (for disease status).
^{b}Optional collection.

TABLE 8
Phase 2a Schedule of Biomarker Sample Collection

| | | Cycle 1 Day | Cycle 2 Day | |
|---|---|---|---|---|
| | Screening | 14 | 14 | Off Treatment |
| DNA | 4 mL blood^{b} | | | |
| Proteomics | 6 mL blood | 6 mL blood | 6 mL blood | 6 mL blood |
| CGH/microarray | Core needle biopsy-flash | | | Core needle |
| | frozen | | | biopsy-flash |
| | | | | frozen (time of |
| | | | | relapse) |
| IHC/FISH | Archived diagnostic | | | Core needle |
| | FFPE tissue | | | biopsy formalin- |
| | Core needle biopsy | | | fixed (time of |
| | formalin-fixed | | | relapse) |
| Tumor Cells^{a} | 2 mL bone marrow | | | |
| | aspirate | | | |

^{a}Bone marrow aspirate samples for analysis should be obtained with the bone marrow biopsies (for disease status).
^{b}Optional collection.

### Drug Concentration Measurements

### Collection of Samples for Analysis

### Blood Samples for ABT-263 Assay

For Phase 1, blood samples for ABT-263 assay were collected by venipuncture into 3-mL evacuated potassium EDTA-containing collection tubes during Cycle 1 at the following times: Day-3, prior to dosing (0 hour) and at 0.5, 1, 2, 3, 4, 6, 8, 24, 48 and 72 (Day 1, pre-dose sample) hours after dosing; Day 1, at 0.5, 1, 2, 3, 4, 6, 8 and 24 (Day 2, pre-dose sample) hours after dosing; Day 14, prior to dosing (0 hour) and at 0.5, 1, 2, 3, 4, 6, 8 hours after dosing. Additional blood samples were collected at 0 hour (pre dose) on Day 14, Cycle 2 through Cycle 6. Sufficient blood were collected to provide approximately 1 mL plasma from each sample. A total of 27 blood samples (approximately 81 mL) were collected per subject for pharmacokinetic analysis during Cycle 1 and one additional blood sample per subject per cycle in the following cycles (up to Cycle 6).

For Phase 2a, blood samples (3 mL) were collected pre-dose (0 hour) and 4 hours post-dose on Cycle 1 Day 14 only.

Blood and plasma samples must be protected from direct sunlight during collection, processing and storage. Immediately after collection, the blood samples were inverted several times to ensure good mixing of the blood and anticoagulant, and were placed in an ice bath.

The timing of blood collections will take priority over all other scheduled study activities except for dosing. The order of blood collections were maintained to the minute such that the time intervals relative to the preceding dosing were the same for all subjects. The time that each blood sample is collected were recorded to the nearest minute.

### Urine Samples for ABT-263 Assay (Phase 1)

Urine for ABT-263 assay were collected in containers without preservatives 0 to 24 hours after dosing on Cycle 1 Day-3 only from the subjects who participate in the Phase 1 dose escalation study. Subjects were instructed to void immediately prior to dosing and one 3 mL aliquot were retained for baseline drug assay (pre-dose sample). Thereafter, urine were collected 0-24 hours following dosing. The start and stop times of the collection interval were recorded to the nearest minute. All urine collected during a collection interval were kept refrigerated until the end of the interval. To ensure complete urine collection, subjects were instructed to void into a container at the conclusion of the collection interval.

### Handling/Processing of Samples

### Blood Samples for ABT-263 Assay

The blood samples for ABT-263 assay were centrifuged within 1 hour of collection at 1200-1500×g for 15 minutes using a refrigerated centrifuge (2-8° C.) to separate the plasma. The plasma samples were transferred using plastic pipettes into screw-capped polypropylene tubes labeled with the drug number name, type of sample (plasma), the protocol number, the subject number, the study cycle and day and the planned time of sampling relative to dosing. The plasma samples were frozen at −20° C. or colder within 1 hour after collection and will remain frozen until shipped.

### Urine Samples for ABT-263 Assay

All urine collected over the specified time interval were thoroughly mixed and the volume were measured and recorded. One 3 mL aliquot were placed in a screw-capped polypropylene tube labeled with the drug number name, type of sample (urine), the protocol number, the subject number, the study cycle and day and the planned collection interval. The urine samples were frozen at −20° C. until shipped.

### Efficacy Variables

All efficacy analyses are exploratory in nature. The exploratory efficacy endpoints include tumor response (determined using IWG Criteria), progression free survival (PFS), time to tumor progression (TTP), overall survival (OS), duration of overall response and ECOG performance status.

IWG Criteria for Tumor Response

Only subjects with measurable disease are eligible for the Phase 2a study. Changes in the measurable lesions over the course of therapy were evaluated using the IWG criteria listed below.

### Eligibility

Only subjects with measurable disease at baseline can have objective tumor response evaluated as an endpoint.

| | |
|---|---|
| Measurable | The presence of at least one measurable lesion. If the |
| Disease | measurable disease is restricted to a solitary lesion, |
| | its neoplastic nature should be confirmed |
| | by cytology/histology. |
| Measurable | Lesions that can be accurately measured in at least one |
| Lesions | dimension with longest diameter ≧10 mm. |

All measurements should be taken and recorded in metric notation using a ruler or calipers. All baseline evaluations should be performed as closely as possible to the beginning of treatment and never more than four weeks before the beginning of the treatment.

The same method of assessment and the same technique should be used to characterize each identified and reported lesion at baseline and during follow-up.

Clinical lesions will only be considered measurable when they are superficial (e.g., skin nodules and palpable lymph nodes). For the case of skin lesions, documentation by color photography including a ruler to estimate the size of the lesion is required.

### Methods of Measurement

CT is the preferred method to measure lesions selected for response assessment. MRI may be used if medically indicated (e.g., severe contrast allergy). Conventional CT and MRI should be performed with cuts of 7 mm or less in slice thickness contiguously. Spiral CT should be performed using a 5 mm contiguous reconstruction algorithm. This applies to tumors of the chest, abdomen and pelvis.

Lesions on chest x-ray are acceptable as measurable lesions when they are clearly defined and surrounded by aerated lung; however, CT is preferable.

For accurate objective response evaluation, ultrasound (US) should not be used to measure tumor lesions. However, US is a possible alternative to clinical measurements of superficial palpable lymph nodes, subcutaneous lesions and thyroid nodules. US might also be useful to confirm the complete disappearance of superficial lesions usually assessed by clinical examination.

Cytology and histology can be used to differentiate between partial response (PR) and complete response (CR) in rare cases (e.g., after treatment to differentiate between residual benign lesions and residual malignant lesions in tumor types such as germ cell tumors).

Assessment of response were performed by the investigator and documented on the appropriate CRF page.

Complete Response (CR) requires the following:
1. Complete disappearance of all detectable clinical and radiographic evidence of disease and disappearance of all disease-related symptoms if present before therapy, and normalization of lymphoma related biochemical abnormalities.
2. All lymph nodes and nodal masses must have regressed to normal size (≦1.5 cm in their greatest transverse diameter for nodes>1.5 cm before therapy). Previously involved nodes that were 1.1 to 1.5 cm in their greatest transverse diameter before treatment must have decreased to ≦1 cm in their greatest transverse diameter after treatment, or by more than 75% in the sum of the products of the greatest diameters (SPD).
3. The spleen, if considered to be enlarged before therapy on the basis of a CT scan, must have regressed in size and must not be palpable on physical examination.
4. For subjects with disease in the bone marrow, tumor infiltrate must be cleared on repeat bone marrow aspirate and biopsy of the same site. The sample on which this determination is made must be adequate (≧20 mm biopsy core).

### Complete Response/Unconfirmed (CRu):

1. CR/unconfirmed (CRu) includes those patients who fulfill criteria 1 and 3 above, but with one or more of the following features:
2. A residual lymph node mass greater than 1.5 cm in greatest transverse diameter that has regressed by more than 75% in the SPD. Individual nodes that were previously confluent must have regressed by more than 75% in their SPD compared with the size of the original mass.
3. Indeterminate bone marrow (increased number or size of aggregates without cytologic or architectural atypia).
   Partial Response (PR) requires the following:
1. A≧50% decrease in SPD of the six largest nodes or nodal masses. These nodes or masses should be selected according to the following features: (a) they should be clearly measurable in at least two perpendicular dimensions, (b) they should be from as disparate regions of the body as possible, and (c) they should include mediastinal and retroperitoneal areas of disease whenever these sites are involved.
2. No increase in the size of the other nodes, liver, or spleen.
3. Splenic and hepatic nodules must regress by at least 50% in the SPD.
4. With the exception of splenic and hepatic nodules, involvement of other organs is considered assessable and not measurable disease.
5. Bone marrow assessment is irrelevant for determination of a PR because it is assessable and not measurable disease; however, if positive, the cell type should be specified in the report, e.g., large-cell lymphoma or low-grade lymphoma (i.e., small, lymphocytic small cleaved, or mixed small and large cells).
6. No new sites of disease. Stable Disease (SD):
   Stable disease is defined as less than a PR (see above) but is not progressive disease (see below).
   Progressive Disease (PD) requires the following (for PR, nonresponders):
1. A≧50% increase from nadir in the SPD of any previously identified abnormal node for PRs or nonresponders.
2. Appearance of any new lesion during or at the end of therapy.
   Relapsed Disease requires the following (for CR, CRu):
1. Appearance of any new lesion or increase by ˜50% in the size of previously involved sites.
2. ˜50% increase in greatest diameter of any previously identified node greater than 1 cm in its short axis or in the SPD of more than one node.
   A summary of the criteria described above is shown below.

| | Physical | | Lymph Node | Bone |
|---|---|---|---|---|
| Response Category | Examination | Lymph Nodes | Masses | Marrow |
| CR | Normal | Normal | Normal | Normal |
| CRu | Normal | Normal | Normal | Indeterminate |
| | Normal | Normal | >75% | Normal or |
| | | | decrease | indeterminate |
| PR | Normal | Normal | Normal | Positive |
| | Normal | ~50% | ~50% | Irrelevant |
| | Decrease in | ~50% | ~50% | Irrelevant |
| | liver/spleen | decrease | decrease | |
| Relapse/progression | Enlarging | New or | New or | Reappearance |
| | liver/spleen; new | increased | increased | |
| | sites | | | |

Note:
See text for definitions of “normal” and “indeterminate.”

### Confirmation

The goal of confirmation of objective response is to avoid overestimating responses. In cases where confirmation of response is not feasible, it should be made clear when reporting the outcome that the response(s) is (are) not confirmed.

To be assigned a status of PR, CR or CRu changes in tumor measurements must be confirmed by repeat assessments that should be performed within 4-8 weeks after the criteria for response are first met.

In the case of SD, follow-up measurements must have met the SD criteria at least once after study entry at a minimum interval, not less than 6 weeks following initiation of treatment.

5.3.5 Pharmacokinetic Variables

Values for the pharmacokinetic parameters of ABT-263, including the maximum observed plasma concentration_{(Cmax)}, the time to_{Cmax}(peak time,_{Tmax)}, the terminal phase elimination rate constant (f3), terminal elimination half-life_{(t1/2)}, the area under the plasma concentration-time curve (AUC) from time 0 to the time of the last measurable concentration (AUCt) and from time 0 to infinite time (AUC∞) for the doses on Cycle 1 Day-3, Cycle 1 Day 1 and Cycle 1 Day 14 in Phase 1, whenever applicable, were determined using noncompartmental methods. The percent of dose recovered in urine as ABT-263 (% Ae) and renal clearance (CLR) were determined if there is meaningful amount of ABT-263 recovered in urine. Additional parameters may be calculated if useful in the interpretation of the data.

5.3.6 Pharmacogenetic Variables

DNA samples may be analyzed for genetic factors contributing to the subject's response to ABT-263 in terms of pharmacokinetics and safety. The samples may also be used for the development of a diagnostic test for such a drug response. Based on observations in rats and the projection of human PK, phase II enzymes, Bcl-2 family members and intestinal transporters may be of primary interest. Genetic studies in general may include determination of the relationship of genetic haplotypes and drug metabolism, transport, therapeutic response and adverse events. The samples may also be used for the development of a diagnostic test for drug response.

### Pharmacodynamic Variables

Several putative biomarkers of efficacy and response were evaluated in this protocol with the goal of defining the relationship between drug concentration and disease status.

Examination of the proteomic profiles of subjects on the ABT-263 clinical trials may reveal patterns of protein/peptide concentrations that may be further evaluated in future clinical studies to determine any prognostic value and any correlation with clinical response. Samples were analyzed for predictive or drug-responsive proteomic markers. In the event that any plasma samples are unused, remaining samples were anonymized and banked for use in diagnostic test development efforts.

Tissue slides from diagnostic biopsies were used for immunohistochemistry (IHC) and fluorescence in situ hybridization (FISH). Immunohistochemical analysis were performed on tissue slides from archived, diagnostic tissue blocks and banked for use in diagnostic test development efforts. Given the targeted nature of ABT-263 for a subset of anti-apoptotic proteins (Bcl-2, Bcl-X_{L}, and Bcl-w), the relationship between sensitivity of cell lines to ABT-263 and the expression levels of the Bcl-2 family members has been examined in human tumor cell lines. Bcl-2 expression levels (mRNA and protein) exhibited strong correlations with sensitivity, and the protein concentrations of Bcl-X_{L} paralleled that of Bcl-2. Conversely, higher expression levels (mRNA and protein) of Mcl-1 was associated with resistance. Taken together, preclinical data suggests that tumor cells sensitive to ABT-263 will exhibit high Bcl-2 and Bcl-X_{L} expression coupled to low Mcl-1 expression whereas the inverse were reflective of ABT-263 resistance. Consequently, pre- and post-treatment subject tumor specimens obtained from diagnostic biopsies (with no treatment intervention) or fixed core needle biopsies and/or bone marrow aspirates/core biopsies were evaluated for relative expression of the Bcl-2 family members. Moreover, an additional pre- and post-treatment biopsy were flash frozen for genetic analysis, which may include expression microarray to assess gene expression and/or CGH to assess global gene amplifications and deletions.

Amplification of 1 8q2 1, containing the Bcl-2 gene locus, has been observed in SCLC cell lines having the highest sensitivity to ABT-263 and represents a potential genetic lesion associated with drug sensitivity. Fluorescent in-situ Hybridization (FISH) were conducted on tissue from archived tumor samples from subjects participating in this study to assess amplifications and translocations in the Bcl-2 gene and other family member genes which may prove to be informative. The potential relationship between amplification of these genes and the clinical outcome in these patients were examined as a subjects stratification tool. Biospecimens collected during the course of this study may be banked and used in the future to investigate new scientific questions related to this study.

### Treatments Administered

Subjects self-administered ABT-263 orally once daily (QD). Each dose were taken with approximately 240 mL of water. On days that pre-dose pharmacokinetic sampling is required (Cycle 1 Day-3, Cycle 1 Day 1, Cycle 1 Day 2, Cycle 1 Day 14, Cycle 2 Day 14, Cycle 3 Day 14, Cycle 4 Day 14, Cycle 5 Day 14 and Cycle 6 Day 14 in Phase 1 and on Cycle 1 Day 14 in Phase 2a) dosing will occur in the morning in the clinic to facilitate pharmacokinetic sampling. In Phase 1, all subjects will receive ABT-263 under fasting conditions (after an 8-hour fast and 4 hours before lunch) on Cycle 1 Day-3 and at 30 minutes after the start of a standard breakfast on Cycle 1 Day 1. On all other dosing days of Phase 1, the subjects will receive ABT-263 at approximately 30 minutes after breakfast. In Phase 2a, all subjects will self-administer ABT-263 at approximately 30 minutes after breakfast. The effect of food on pharmacokinetics were evaluated and changes were initiated if fasting conditions are superior.

On extensive PK collection days (Cycle 1 Day-3, Cycle 1 Day 1 and Cycle 1 Day 14 of Phase 1), the time of each drug administration were recorded to the nearest minute. On all other days, subjects were instructed to record the date and time they take their study drug.

Subject diaries were provided by Abbott.

Identity of Investigational Products

| Study Drug | Formulation |
|---|---|
| ABT-263 | Powder for Oral |
| | Solution (2.0 |
| | grams/bottle base |
| | equivalent, |
| | 25 mg/mL when |
| | mixed) |

* N/A = Not Applicable

### Diluents for Constitution

Phosal® 53 Medium Chain Triglyceride (MCT), 120 grams/bottle.
Alcohol (Ethanol), Dehydrated, USP/EP/JP 200 Proof

### Phase 1/2a Study Pharmacokinetics

ABT-263 exposure is dose-proportional
   Mean AUC: 47 μg h/mL at 225 mg (n=4); 100 μg·h/mL at 315 mg (n=5); 109 μg·h/mL at 440 mg (n=3)
   Preclinical target AUC: 53 to 88 μg·h/mL·ABT-263 peak concentration (Cₘₐₓ) ˜8 hours post-dose
Half-life ˜14-20 hours
Peak-to-trough plasma concentration ratio ˜3 fold at steady state
Inter-subject variability in exposure ˜40%
Food has mild positive effect on ABT-263 oral absorption
   Additional data shown in attached figures.

The foregoing is meant to illustrate the invention but not to limit it. Variations and changes obvious to one skilled in the art are intended to be within the scope of the invention as defined in the appended claims.

We claim:

1. A composition for oral administration comprising N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide, Phosal® 53 Medium Chain Triglyceride and dehydrated ethanol.
